(19) United States
(12) Patent Application Publication
(10) Pub. No.: US 2020/0095389 A1
(43) Pub. Date: Mar. 26, 2020

(54) PHYSICAL DEPOSITION OF SILICEOUS PARTICLES ON PLASTIC SUPPORT TO ENHANCE SURFACE PROPERTIES

(71) Applicant: MATHILDE GOSSELIN, GRANBY (CA)

(72) Inventors: MATHILDE GOSSELIN, GRANBY (CA); CHARLES GAUDREAULT, FORESTVILLE (CA); HAKIM RAHMA, GRANBY (CA); HUBERT CABANA, MAGOG (CA)

(21) Appl. No.: 16/483,834

(22) PCT Filed: Feb. 6, 2018
(86) PCT No.: PCT/CA2018/050131

§ 371 Date: Aug. 6, 2019

Related U.S. Application Data
(60) Provisional application No. 62/455,277, filed on Feb. 6, 2017.
(60) Provisional application No. 62/474,111, filed on Mar. 21, 2017.
(60) Provisional application No. 62/598,993, filed on Dec. 14, 2017.

(51) Int. Cl. C08J 7/06; C07K 17/14; C12N 11/14; C08K 3/36; C08K 9/02; C08K 9/04

(52) U.S. Cl. CPC C08J 7/06; C07K 17/14; C12N 11/14; C08K 3/36; C08K 9/02; C08K 2201/011; B09C 2101/00; C08J 2300/22; C08J 2323/06; C08K 2201/003; C08K 9/04

(57) ABSTRACT

The present invention relates to products and method of preparing and using surface modified polymeric material having siliceous particles deposited thereon. The method and article are disclosed wherein a plastic substrate is provided with high surface area and increase of surface roughness. The methods for treating the surface are provided.

### CROSS-REFERENCE TO RELATED APPLICATIONS

This application claims priority of U.S. patent application No. 62/455,277 filed on Feb. 6, 2017, U.S. provisional patent application No. 62/474,111 filed on Mar. 21, 2017, and U.S. provisional patent application No. 62/598,993 filed on Dec. 14, 2017, the specifications of which are hereby incorporated by reference in their entireties.

### BACKGROUND

### (a) Field

The invention relates to surface modified polymeric material, and method of preparing and using the same. More specifically, the subject matter relates to surface modified polymeric material comprising a plurality of silica particles deposited and partially embedded on a surface thereof.

### (b) Related Prior Art

Surface modification of substrates by silanization is widely used to give to a support new properties by changing the physicochemical properties of the original support. It can be used to change its topography, change the surface tension, protect the product from alteration and so on. The silanization process is carried out by a variety of methods including sol-gel process (e.g. US patent application no. 2013/0236641), sputter deposition (e.g. U.S. Pat. No. 5,616,369), electron beam deposition (e.g. US patent application no. 2011/0116992), and plasma enhanced chemical vapor deposition (e.g. U.S. Pat. No. 4,096,315 and US patent application no. 2010/0098885A1).

The reaction of silanization on hydrophilic substrates is achieved through the hydroxyl groups to form a polysiloxane network. Indeed, the presence of polar chemical functions can serve as anchoring points for the polysiloxane formation. Some functional silanes are sometimes used to introduce silanol groups to initiate the silanization process. However, the hydrophobic supports modification requires an oxidation reaction that involves the use of expensive equipment and toxic chemicals (Gutowski, W. S. et al., “Surface silanization of polyethylene for enhanced adhesion”, J. Adhesion 43:139-155 (1993)). Moreover, this process is not ideal to bring surface roughness and surface area which are very helpful for surface adhesion.

An alternative to silanization is to use inorganic filler for the purpose of changing the surface roughness and wettability of the support matrix. The loading percentage is usually high enough to change the mechanical and physical properties of the support material. Thus, changing the surface properties also changes the characteristics of the material (e.g. mechanical strength, density of the material, etc.) which can negatively affect the product performance in some applications.

Another strategy to modify a substrate surface while keeping a high surface roughness and surface area, is the deposition of inorganic or hybrid particles on supports. It was shown that it is possible to create a covalent linking between the support and the inorganic particles by thermal treatment (e.g. U.S. Pat. No. 8,153,249). This method requires high temperature and is mostly applied to the inorganic supports. Moreover, the process is applicable for small size particles (below 1 μm). However, surface modification of polymers at very high temperature causes their destruction. Also, it would be difficult to create a covalent bonding between hydrophobic plastics and the inorganic particles.

Therefore, silanization, filler addition and particles deposition have limitations of their own. Therefore, there is a need for alternative methods of surface modification with silica particles or capsules. The methods presented herein propose surface modification by deposing or embedding siliceous particles or capsules on polymeric surfaces without changing the intrinsic properties of the polymeric support.

### SUMMARY

According to an embodiment, there is provided a surface modified polymeric material comprising a plurality of silica particles deposited and partially embedded on a surface thereof, wherein the silica particles are bioavailable for interaction with a microorganism or a biological molecule or complex, available for chemical interaction, available for chemical reaction, or a combination thereof.

The plurality of silica particles may be a plurality of one type of silica particle, a plurality of at least one type of silica particle, or a plurality of more than one type of silica particle.

The polymeric material may be a plastic material.

The plurality of silica particles deposited and partially embedded on a surface thereof may be deposited on the surface at or over a melting point of the polymeric material.

The silica particles may be about 10% to about 90% partially embedded in the polymeric material.

The silica particles cover from about 0.01% to 100% of the surface.

The silica particle may be a nanoparticle, a microparticle, a nanosphere, a microsphere, or combinations thereof. The silica particles have a diameter of from about 10 nm to about 15 mm or a combination thereof. The silica particles may be crystalline silica or amorphous silica. The silica particles may be spherical particles or of a random geometry. The silica particles may be hollow particles or full particles. The silica particles may be porous or non-porous. The silica particles may comprise a chemical functional group. The chemical functional group may be available for the chemical reaction and/or chemical interaction.

The silica particles may be covered with an allotrope of carbon.

The silica particles may be covered with metallic particles or a coating.

The coating may be a metals salt coating, a metal oxide coating, an organometallic coating, an organic coating.

The organic coating may be a polymer, a biopolymer or a combination thereof.

The silica particles may be covered or coated with a microorganism.

The microorganism may be a bacteria, a fungi, a yeast, a mold, a spore, a filament, a gram negative bacteria, a gram positive bacteria, a dried microorganism, a microfilm supporting microorganism in a growth ready state, a vegetative state microorganism.

The vegetative state microorganism may be synchronized and arrested in a specific phase of life cycle, arrested in a specific phase of life cycle, not synchronized and arrested in a specific phase of life cycle, not synchronized in a specific growth phase, ready to be activated in the presence of a suitable carbon source, or a combination thereof.

The silica particles have encapsulated, adsorbed and/or absorbed a chemical, a biologically active molecule, or a combination thereof.

The biologically active molecule comprises an enzyme, a hormone, an antibody or a functional fragment thereof, a bio suppressant, or combinations thereof.

The chemical comprises an antibiotic, an anti-viral, an anti-toxin, a pesticide, or combinations thereof.

The silica particles may be a silica shell having a thickness of from about 50 nm to about 500 μm, and a plurality of pores, the shell forming a capsule having a diameter from about 0.2 μm to about 1500 μm, and having a density of about 0.01 g/cm³ to about 1.0 g/cm³,

wherein the shell comprises from about 0% to about 70% Q3 configuration, and from about 30% to about 100% Q4 configuration, or
wherein the shell comprises from about 0% to about 60% T2 configuration and from about 40% to about 100% T3 configuration, or
wherein the shell comprises a combination of T and Q configurations thereof, and
wherein an exterior surface of the microcapsule may be covered by a functional group.

The shell comprises about 40% Q3 configuration and about 60% Q4 configuration, or about 100% Q4 configuration.

The pores have pore diameters from about 0.5 nm to about 100 nm.

The surface modified polymeric material silica particles may comprise a surface layer.

The surface layer comprises a thickness from about 1 nm to about 10 nm.

The surface layer may be functionalized with an organosilane.

The the organosilane may be chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

The surface layer may be functionalized with a hydroxyl group, an amino group, a benzylamino group, a chloropropyl group, a disulfide group, an epoxy group, a mercapto group, a methacrylate group, a vinyl group, and combinations thereof.

According to another embodiment, there is provided a product prepared with the surface modified polymeric material of the present invention.

The product may be a sheet of polymeric material, a polymeric material droplet or bead, a polymeric material media for use in wastewater treatment.

The product may have one or more surface of the product comprises the plurality of silica particles deposited and partially embedded thereof.

According to another embodiment, there is provided a process for the preparation of a surface modified polymeric material comprising a plurality of silica particles deposited and partially embedded on a surface thereof, the process comprising the step of:

contacting a surface of polymeric material at a temperature at or over a melting temperature of the polymeric material with a plurality of silica particles, wherein the silica particles are deposited and partially embedded thereon, and are bioavailable for interaction with a microorganism or a biological molecule or complex, available for chemical interaction, available for chemical reaction, or a combination thereof.

The plurality of silica particles may be a plurality of one type of silica particle, a plurality of at least one type of silica particle, or a plurality of more than one type of silica particle.

The polymeric material may be a plastic material.

The silica particles may be deposited and partially embedded in the polymeric material by a mechanical treatment, thermal treatment, chemical treatment, or a combination thereof.

The silica particles may be deposited and partially embedded during the polymeric material production process by an extrusion process, an injection process, a thermoforming, a compression molding, a rotational molding, a blow molding, a pultrusion, or combinations thereof.

The polymeric material may be provided as droplets.

The silica particles may be deposited and partially embedded after the polymeric material production process.

The silica particles may be deposited and partially embedded in the plastic using heat supplied by convection, conduction or radiation.

The polymeric material may be heated to a temperature at or over a melting temperature of the polymeric material provided by a hot air or gas, a flame, a hot slurry, a hot liquid, a sonication, a mechanical wave, a plasma, an electricity, a lamp, a heating element, a conductive plate, or combinations thereof.

The silica particles may be deposited or partially embedded as a suspended powder, as a slurry, or a combination thereof.

The silica particles may be about 10 to about 90% partially embedded in the polymeric material.

The silica particles cover from about 0.01% to 100% of the surface.

The silica particles may be a nanoparticle, a microparticle, a nanosphere, a microsphere, or combinations thereof. The silica particles have a diameter of from about 10 nm to about 10 mm or a combination thereof. The silica particles may be crystalline silica or amorphous silica. The silica particles may be spherical particles or of a random geometry. The silica particles may be hollow particles or full particles. The silica particles may be porous or non-porous. The silica particles comprise a chemical functional group. The chemical functional group may be available for the chemical reaction. The silica particles may be covered with an allotrope of carbon. The silica particles may be covered with metallic particles or a coating. The coating may be a metals salt coating, a metal oxide coating, an organometallic coating, an organic coating. The organic coating may be a polymer, a biopolymer or a combination thereof.

The silica particles may be covered or coated with a microorganism.

The microorganism may be a bacteria, a fungi, a yeast, a mold, a spore, a filament, a gram negative bacteria, a gram positive bacteria, a dried microorganism, a microfilm supporting microorganism in a growth ready state, a vegetative state microorganism.

The vegetative state microorganism may be synchronized and arrested in a specific phase of life cycle, arrested in a specific phase of life cycle, not synchronized and arrested in a specific phase of life cycle, not synchronized in a specific growth phase, ready to be activated in the presence of a suitable carbon source, or a combination thereof.

The silica particles have encapsulated, adsorbed or absorbed a chemical, a biologically active molecule, or a combination thereof.

The biologically active molecule comprises an enzyme, a hormone, an antibody or a functional fragment thereof, a bio suppressant, or combinations thereof.

The chemical comprises an antibiotic, an anti-viral, an anti-toxin, a pesticide, or combinations thereof.

The silica particles may be a silica shell having a thickness of from about 50 nm to about 500 μm, and a plurality of pores, the shell forming a capsule having a diameter from about 0.2 μm to about 1500 μm, and having a density of about 0.01 g/cm³ to about 1.0 g/cm³,

wherein the shell comprises from about 0% to about 70% Q3 configuration, and from about 30% to about 100% Q4 configuration, or
wherein the shell comprises from about 0% to about 60% T2 configuration and from about 40% to about 100% T3 configuration, or
wherein the shell comprises a combination of T and Q configurations thereof, and
wherein an exterior surface of the microcapsule may be covered by a functional group.

The shell comprises about 40% Q3 configuration and about 60% Q4 configuration, or about 100% Q4 configuration.

The pores have pore diameters from about 0.5 nm to about 100 nm.

The silica particles may further comprising a surface layer.

The surface layer comprises a thickness from about 1 nm to about 10 nm.

The surface layer may be functionalized with an organosilane.

The organosilane may be chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

The surface layer may be functionalized with a hydroxyl group, an amino group, a benzylamino group, a chloropropyl group, a disulfide group, an epoxy group, a mercapto group, a methacrylate group, a vinyl group, and combinations thereof.

According to another embodiment, there is provided a method for the treatment of wastewater or a contaminated soil, comprising contacting wastewater or contaminated soil with a surface modified polymeric material of the present invention, a product according to the present invention, or combinations thereof, for a time sufficient and under conditions sufficient for decontaminating the wastewater or contaminated soil.

The treatment of wastewater may be in a moving bed biofilm reactor (MBBR), an Integrated Fixed-Film Activated Sludge (IFAS) reactor, an aerated pond, a non-aerated pond, a membrane bioreactor (MBR), a sequential batch reactor (SBR), a water polishing processes, with an activated sludge, or combinations thereof.

The surface modified polymeric material or the product may be a media for use in wastewater treatment.

According to another embodiment, there is provided biological process comprising contacting a culture media with a surface modified polymeric material of the present invention, a product according to of the present invention, or combinations thereof for a time sufficient and under conditions sufficient for any one of a fermentation, a pre-culture, a media preparation, a harvesting of a product, concentration of a product, purification of a product.

According to another embodiment, there is provided process comprising contacting a solution with a surface modified polymeric material of the present invention, a product of the present invention, or combinations thereof under conditions sufficient to perform a reaction or an interaction with the surface modified polymeric material and/or the product.

The process may be performed in a column. The process may be a chromatography, an adsorption, a catalysis, or combinations thereof. The process may be an enzymatic process.

The following terms are defined below.

The terms “silica particle(s)” is intended to mean particles from a wide range of silica containing material. The size of the siliceous/silica particles may range from about 10 nm to about 15 mm but may generally be in the range about 1 to about 100 μm. The silica particle may have any geometry and/or they may be spherical. Only one type of silica particle may be used, or a combination of different particles may be used for the coating. The particles may also have adsorbed, encapsulated, absorbed or covalently attached substance. The silica particles may be pure silica, organosilica, or a silica containing material. Therefore, the word “silica” used herein, it may refer to pure silica particle or to particle containing silica and other elements or compounds.

The term “biological molecule” is intended to mean large macromolecules (or polyanions) such as proteins, carbohydrates, lipids, and nucleic acids, as well as small molecules such as primary metabolites, secondary metabolites, and natural products. A more general name for this class of material is biological materials. According to an embodiment, the biological molecule may be a complex of several molecules, such as for example an enzyme and a substrate, an antibody and a bound target, a receptor and a ligand, of generally proteins interacting together, and/or enzyme interacting together.

The term “polymeric material” in intended to mean any polymer or composite thereof that may be heated to or over its melting point and on which silica particles may be deposited. According to an embodiment, the polymeric material is a plastic material or a composite thereof. The geometry or the shape of the polymeric material may be variable, since the siliceous deposition technology can be applied to any plastic surface.

In the present document, as known in the art, the condensed siloxane species, the silicon atoms through mono-, di-, tri-, and tetra-substituted siloxane bonds are designated as Q1, Q2, Q3, and Q4, respectively. Similarly, the condensed organosilane with mono-, di-, and tri-substituted siloxane bonds are designated as T1, T2, T3, respectively.

### BRIEF DESCRIPTION OF THE DRAWINGS

Further features and advantages of the present disclosure will become apparent from the following detailed description, taken in combination with the appended drawings, in which:

FIG. 1 illustrates a polymeric material on which porous silica microspheres according to U.S. Pat. No. 9,346,682 have been deposited, according to an embodiment of the present invention;

FIG. 2A is a scanning electronic microscope image at 92× of a high-density polyethylene (HDPE) material without silica particles;

FIG. 2B. is a scanning electronic microscope image at 67× of a high-density polyethylene (HDPE) material with particles according to U.S. Pat. No. 9,346,682 deposited on its surface according to an embodiment of the present invention;

FIG. 3A is a photograph of a high-density polyethylene (HDPE) plastic media used for water treatment subjected to a thermal treatment without silica particles deposition;

FIG. 3B is a photograph of a high-density polyethylene (HDPE) plastic media used for water treatment subjected to a thermal treatment with particles according to U.S. Pat. No. 9,346,682 deposited on its surface according to an embodiment of the present invention;

FIG. 3C is a photograph of two plastic media. The plastic media on the left is from the media shown in FIG. 3A and the media on the right is from the media shown in FIG. 3B, and shows the morphological difference between the two-plastic media with and without the silica coating.

FIG. 4A illustrates the microbial count over time from a lab scale test related to oil sand wastewater treatment. The triangle shows the microbial count for plastics media without silica particles deposition. The square shows the count for plastics media with 5 μm silica microspheres according to U.S. Pat. No. 9,346,682 deposited on its surface according to an embodiment of the present invention. The circle shows the microbial count for plastics media with 20 μm silica microspheres according to U.S. Pat. No. 9,346,682 deposited on its surface according to an embodiment of the present invention;

FIG. 4B shows the remaining naphthenic acid (NA) from oil sand wastewater after biological treatment. Four treatments are shown; the first is the control where only activated sludge was used; the second treatment consist of activated sludge and regular plastic media; the third treatment consist of activated sludge and plastic media covered with 5 μm silica microspheres according to U.S. Pat. No. 9,346,682 deposited on its surface according to an embodiment of the present invention; the fourth treatment consist of activated sludge and plastic media covered with 20 μm silica microspheres according to U.S. Pat. No. 9,346,682 deposited on its surface according to an embodiment of the present invention;

FIG. 5 shows the results of chemical oxygen demand in the effluent of two pilot moving bed biofilm reactor (MBBR) in a test related to municipal wastewater. The dashed line represents the results of a MBBR reactor using plastic media without silica deposition. The continuous line represents the results of a MBBR reactor using plastic media with silica microspheres according to U.S. Pat. No. 9,346,682 deposited on its surface according to an embodiment of the present invention;

FIG. 6 shows the results of thiocyanate decrease over time due to its consumption by bacteria immobilized on plastic media. At time zero, the wastewater influent contains about 250 ppm thiocyanate (100%). Two conditions are tested in parallel. The first condition, continuous line, refer to a test done using traditional plastic media. The second condition, dashed line, refer to a test done using plastic media coated with silica according to U.S. Pat. No. 9,346,682 deposited on its surface, according to an embodiment of the present invention.

FIG. 7A shows the results of conversion of ABTS to a colored product by a laccase enzyme immobilized on a plastic media coated with silica microspheres according to an embodiment of the present invention; 1) shows 5 falcons tube, each containing a plastic media. At time zero conversion is starting and can be observed at the surface of the plastic media. Note that the tube at the far right is a control containing a plastic media without silica particle. 2) shows the same tube after 30 minutes. Note that the control tube is missing. 3) shows the five tubes after 4 hours.

FIG. 7B show the absorbance measurement of an ABTS solution being catalyzed by a laccase enzyme immobilized on a plastic media coated by silica. The experiment is monitored over a period of 26 hours.

FIG. 8 show the results of the adsorption of 16 emergent contaminants at a concentration of 100 μg/L in 60 mL with 1 media at pH 6.5. These results have been compared with silica microsphere alone at a concentration of 10 g/L.

FIG. 9A is a scanning electronic microscope image at 351× of a high-density polyethylene (HDPE) material without silica particles and the EDX analysis.

FIG. 9B is a scanning electronic microscope image at 427× of a high-density polyethylene (HDPE) material with non-spherical silica particles deposited on its surface and EDX analysis.

### DETAILED DESCRIPTION

The present invention concerns polymeric material covered with siliceous particles or capsules by a physical deposition method. The present invention concerns both the siliceous particles covered polymeric material and the deposition method.

The impact of the invention is to provide a method of siliceous particles or capsules deposition on polymeric material. The invention depicts the types of siliceous particles that can be used and the description of the processes. Mainly the methods discussed below are thermal, the use of high thermal process may lead to the decomposition of the polymeric material. The thermal deposition may be done during the extrusion process, injection process, after the production after melting of the polymeric material.

In another embodiment of the invention, the plastic media coated with siliceous particles is further modified with the addition by adhesion, adsorption, absorption chemical reaction or immobilization of various substance such as, but not limited to, microorganisms, virus, enzymes, biomolecules, nutrients, oils, chemical reagent, chemical function, metals, metal oxides, metal salts, inorganic salts, graphene, graphene oxide, other carbon allotropes, or combinations thereof.

The polymeric material may be of any dimension and made of any type of polymer or composite. The geometry or the shape of the plastic material is not important since the siliceous deposition technology can be applied to any plastic surface.

The siliceous/silica particles or capsules may come from a wide range of silica containing material. The surface coverage may range from about 0.01% to about 100%. The size of the siliceous particles may range from about 10 nm to about 15 mm but may generally be in the range about 1 to about 100 μm. A combination of different particles sizes may be used for the coating. The particles may also have adsorbed, encapsulated, absorbed or covalently attached substance. The siliceous-plastic may have any geometry.

The particle described herein refer to siliceous particles in general. It may be pure silica, organosilica, or a silica containing material. Therefore, the word “silica” used herein, it may refer to pure silica particle or to particle containing silica and other elements or compounds.

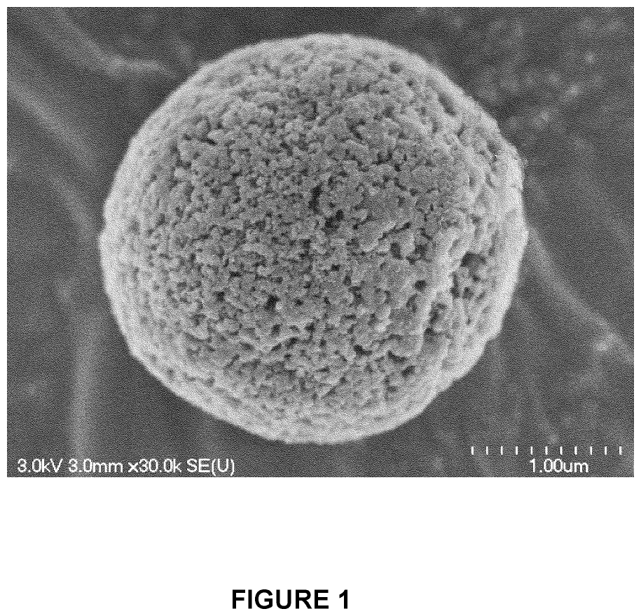

The silica particles or capsules range from about 10 nm to about 15 mm but may generally be in the range about 1 μm to about 100 μm may be made from crystalline silica or from amorphous silica. The particles may be spherical or random shape. The particles may be solid or hollow. It may be porous or non-porous. It may have chemical function such as, but not limited to alkyl chain, chloroalkyl, bromoalkyl, iodoalkyl, hydroxyl, amine, mercapto, epoxy, acrylate, phenyl, benzyl, vinyl, benzylamine, disulfide, quaternary ammonium salt, or combinations thereof. The silica particles may be covered with carbon allotropes including graphite, graphene, carbon nanofibers, single wall carbon nanotubes, multiple wall carbon nanotubes, C60, C70, C76, C82 and C84 fullerenes, etc., and their combination. The silica particles may be combined with, metals, metal oxides, metal salts, inorganic salts or combinations thereof. The silica particle could be a silica capsule. Silica particles used may be for example the hollow porous microsphere disclosed in U.S. Pat. No. 9,346,682 and international patent application publication WO2015135068A1 (e.g. FIG. 1).

The silica particles or capsules may hold microorganisms, virus, enzymes, biomolecules, nutrients, food additives, pharmaceutical active drug, oils, essential oil, a phase change material (PCM) a fragrance, a humidifier, an explosive, a colorant, an insecticide, an herbicide, a fungicide, chemical reagent, chemical function, metals, metal oxides, metal salts, inorganic salts, graphene, graphene oxide, other carbon allotropes or combinations thereof.

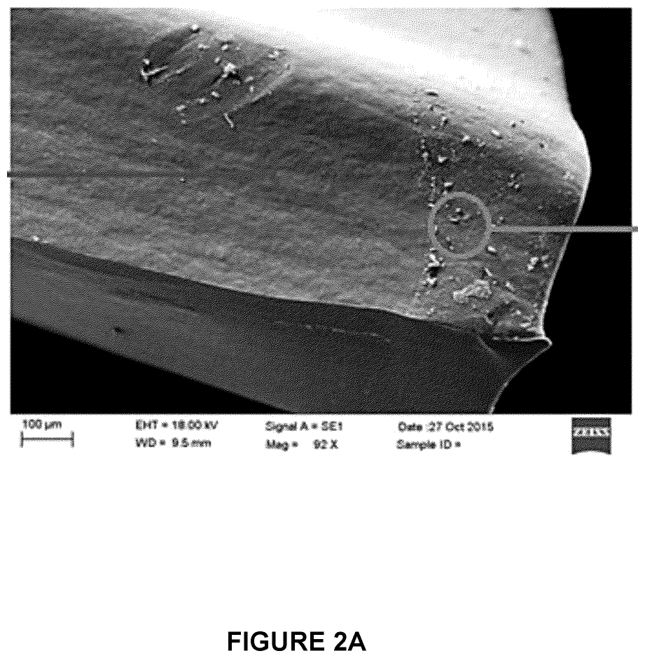

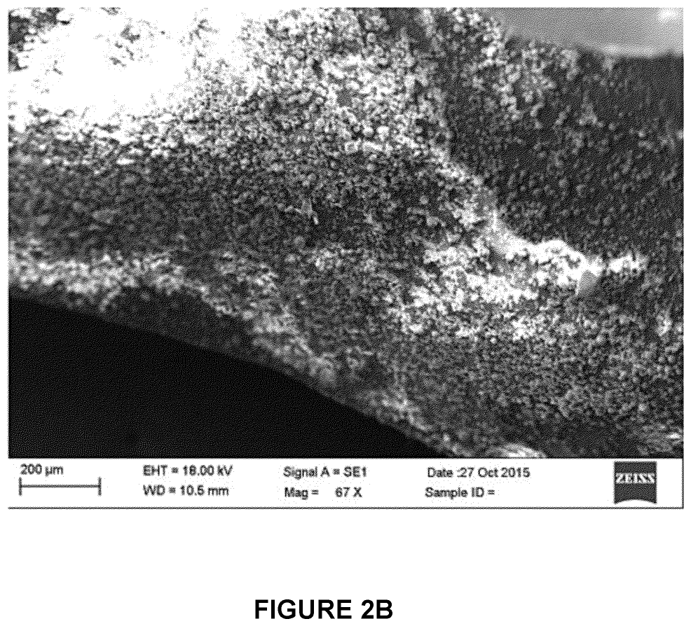

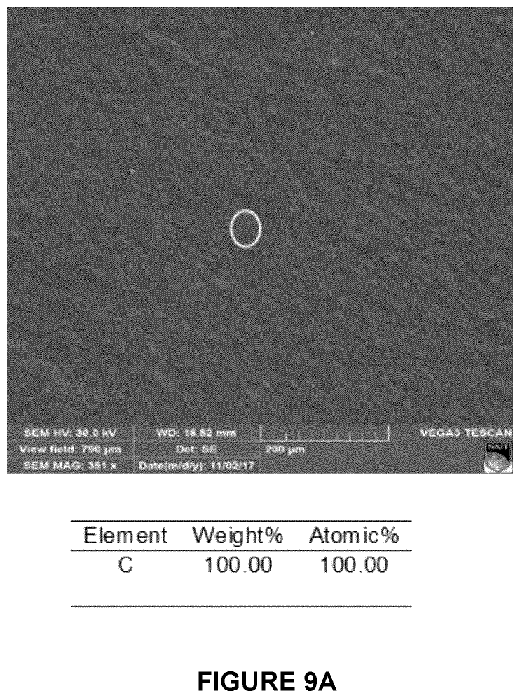

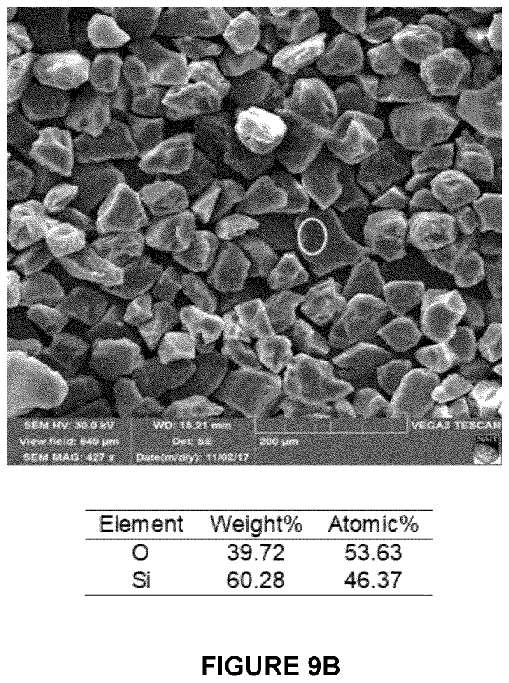

According to an embodiment, the deposition of silica particles or capsules may be performed by thermal processes. According to an embodiment of the thermal process comprises bringing the material to its melting point temperature or above, before exposing the polymeric material to a powder of silica particles. At such temperature, the silica particles sink into the polymer. When the temperature is lowered below the melting temperature, the plastic hardens, and the silica particles becomes embedded on the polymeric support. The resulting polymeric material product has silica particles that are permanently attached to its surface. Scanning electronic microscopy is used to confirm that plastic (FIG. 2A, FIG. 9A) surfaces radically changes when silica particles are deposited (FIG. 2B, FIG. 9B).

In an embodiment, the thermal deposition is performed by applying a flow of silica particles dust in suspension in the atmosphere at any moment when the polymeric material is at or above melting temperature. In another embodiment, the polymeric material is contacted with a hot slurry of silica particles. The polymeric material can be at its melting temperature or above and contacted with a slurry at, above, or under the melting temperature of the polymer.

In another embodiment, the thermal deposition can also be performed during the plastic extrusion process. When the molten plastic exits the extruder through the die, it may be exposed to an atmosphere of silica particles in suspension. Alternatively, instead of an atmosphere, silica particles may be sprayed directly on the molten plastic during the cooling process or during the die extrusion. Alternatively, silica particles may be deposited on the plastic during the extrusion process by pumping silica slurry through a nozzle; where the nozzle would be part of the extrusion die. Alternatively, the polymeric material could be placed in a hot silica particles slurry. The hot slurry can be used to place the plastic in contact with the silica particles, such slurry would be at temperature at or above the plastic melting point. The hot slurry could also be used to perform the silica particles deposition at the same time as the hardening.

In another embodiment, droplets of melted plastic are brought to a slurry of silica particle. As an example, melted plastic droplets may come out from a nozzle and fall down into a slurry of silica particles. The slurry could be at a temperature above, under or at the melting temperature of the plastic depending on the type of process. It could be done in a continuous process were the slurry temperature is under the melting point of the plastic. Alternatively, it could be done in a batch process, such as in a stirred tank, were the slurry temperature is initially above the plastic melting point, the temperature would be lower over time. Alternatively, the process could be done continuously, with a temperature that changes from above to under the plastic melting point across the equipment length.

Thermal deposition can alternatively, be done during or after the plastic injection process. Molten plastic can be injected in a silica slurry in form of droplet. Hardening would occur in the slurry, trapping silica particles on the plastic surface.

Thermal deposition can alternatively be done during or after thermoforming, compression molding, rotational molding, blow molding, filament winding, resin transfer molding (RTM), reaction injection molding (RIM), drape forming or pultrusion.

Deposition of silica particles after the plastic production is also possible. This alternative requires the use of heat to melt the plastic surface in order to do the silica deposition. The heat could be supplied by conduction, convection or radiation. The heat could be generated and or transferred to the plastic by various mean such as, but not limited to: hot gas, flame, hot slurry, hot liquid, sonication, mechanical wave, laser, lamps, heating elements, hot conductive plate, plasma and electricity.

Deposition of silica particles after the plastic production is also possible. If the plastic material has a weight and a geometry that makes fluidization possible, in liquid or in air, fluidization may be an option. A fluid bed could be operated at temperature at or above the melting point of the plastic. Hot air or a hot slurry containing the silica particles could be recirculated in the fluids bed as the fluidization medium.

Other alternatives are possible to do the deposition after plastic production. When the geometry of the plastic makes it possible, the plastic material may be placed in a drum dryer. The dryer temperature could be set at a temperature close to its melting temperature and air containing silica particles dust may be recirculated in the drying chamber. Alternatively, the air temperature could be cycled over and under the melting point to minimize plastic deformation.

Alternatively, if the plastic material is in the form of sheet, it would be possible to blow hot air on its surface to melt the plastic surface. Immediately after, silica particles could be sprayed on the plastic surface. Instead of blowing hot air, infrared radiation may be used to melt the plastic surface before spraying silica particles.

According to an embodiment, the slurry used to transport the silica particles, it could be water, oil or a solvent of organic or inorganic composition.

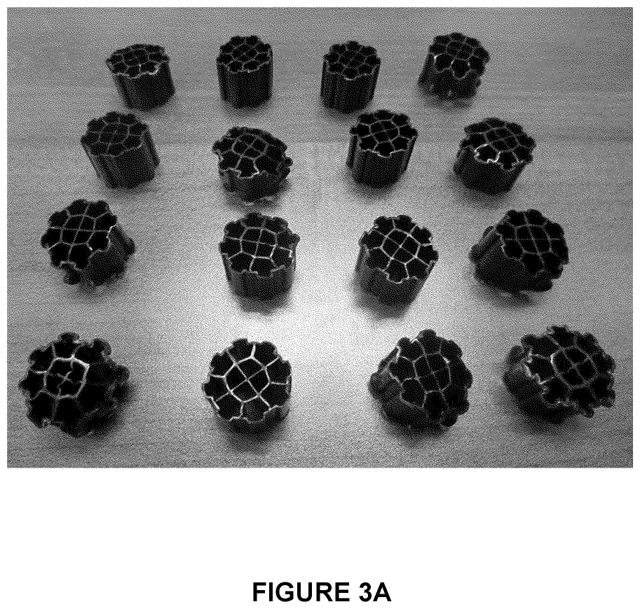

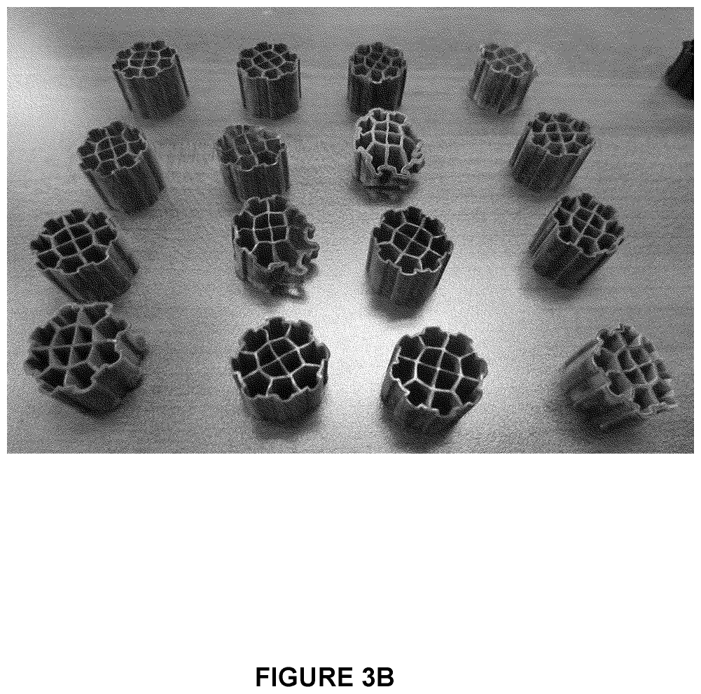

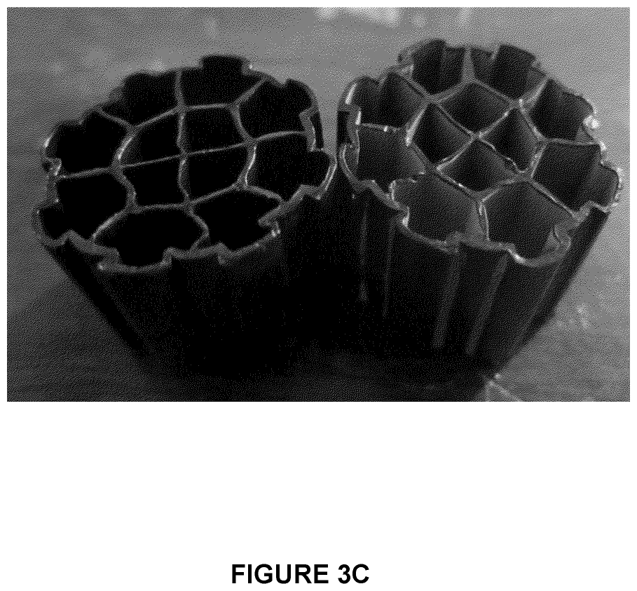

According to an embodiment, the plastic material geometry may be in the form of sheets, thin film or in more complex form. A more complex form could be for example a plastic media used for wastewater treatment such as use in moving bed biofilm reactor (MBBR). High density polyethylene media used for MBBR (FIG. 3A) may be a good candidate for silica particles deposited plastic material (FIG. 3B). In another embodiment of the invention, the plastic media coated with siliceous particles is further modified with the addition by adhesion, adsorption, absorption chemical reaction or immobilization of various substance such as, but not limited to, microorganisms, virus, enzymes, biomolecules, nutrients, oils, chemical reagent, chemical function, metals, metal oxides, metal salts, inorganic salts, graphene, graphene oxide, other carbon allotropes or combinations thereof.

In one embodiment of the invention, the plastic media coated with siliceous particles is further modified with addition, immobilization, or adsorption of microorganisms such as bacteria or fungi such as yeast, mold or a combination of the three. Suitable bacterial species which can be used with the present invention may be chosen from but not limited to the following genera, Pseudomonas, Rhodopseudomonas, Acinetobacter, Mycobacterium, Corynebacterium, Arthrobacterium, Bacillius, Flavorbacterium, Nocardia, Achromobacterium, Alcaligenes, Vibrio, Azotobacter, Beijerinckia, Xanthomonas. Nitrosomonas, Nitrobacter, Methylosinus, Methylococcus, Actinomycetes and Methylobacter, etc. Suitable fungi such as yeast can be chosen from but not limited to the following genera: Saccaromyces, Pichia, Brettanomyces, Yarrowia, Candida, Schizosaccharomyces, Torulaspora, Zygosaccharomyces, etc. Suitable fungi such as mold can be chosen from but not limited to the following genera: Aspergillus, Rhizopus, Trichoderma, Monascus, Penicillium, Fusarium, Geotrichum, Neurospora, Rhizomucor, and Tolupocladium. The plastic media holding microorganism can be further dried stored and re-incubated when needed.

In one embodiment of the invention, the plastic media coated with silica is further modified with addition, immobilization, or adsorption of enzymes. Suitable enzymes can be chosen from but not limited to the following classes, oxidoreductases, transferases, hydrolases, lyases, isomerases, ligases, polymerases. Example are amylase, lipase, protease, esterase, etc.

In one embodiment of the invention, the plastic media coated with silica including particles of one size or a of a combination of different sizes, spherical or of irregular shapes, may be placed in a favorable environment for microorganism's growth, promoting growth onto the media. Then the media could be harvested and dried for further application. The favorable environment could be a bioreactor, a wastewater treatment unit or any other system promoting bacteria growth known to the art. The further applications could be wastewater treatment, biological remediation, industrial biotechnology or any other application known in the art where microorganism's activity is required. The dried plastic media holding the microorganisms can be re-incubated with new batches in order to inoculate the new plastic media.

According to another embodiment, the, plastic coated with silica particles or capsules, spherical or irregular shape, including particles of one size or a combination of different sizes, according to the present invention can be used in many different areas. In wastewater treatment, plastic media coated with silica could be used in, but not limited to: moving bed biofilm reactor (MBBR), integrated Fixed-Film Activated Sludge (IFAS) reactor, aerated and non-aerated pond, membrane bioreactor (MBR), activated sludge processes, sequential batch reactor (SBR), anaerobic digestion process, upflow anaerobic sludge blanket process, biogas production process, ANAMMOX process, water polishing process. The condition modes used in these processes could be aerobic, anaerobic, anoxic or aerobic/facultative anaerobic. In bioprocesses, plastic media coated with silica could be used in, but not limited to: upstream bioprocessing, including and not limited to fermentation, pre-culture, media preparation and harvesting; downstream bioprocessing, including and not limited to concentration and purification. The silica coated plastic media of the present invention could be used to grow bacteria and biofilm such as in bioprocesses and in wastewater treatment; biofilm may be later dried on the plastic media for further applications. The silica coated on the plastic may promote faster biofilm regrowth after biofilm sloughing. In pharmaceutical processes, plastic media coated with silica could be used in but not limited to: active product ingredient manufacturing, purification and concentration. In chemical processes, plastic media coated with silica could be used in but not limited to: adsorption and catalysis reactor. In soil treatment and bioremediation, plastic media coated with silica could also be used.

In one embodiment of the invention, the plastic media coated with silica has an application in stripping process. The packing plastic modified with silica allows the minimization of flow and achieves an efficient separation. Indeed, it increases the surface area and the flow area.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

The present invention will be more readily understood by referring to the following examples which are given to illustrate the invention rather than to limit its scope.

### Example 1

### Silica Particles Deposition after Plastic Material Production

An example of silica particles deposition. A thermal treatment is performed on plastic media (FIG. 3A) of high density polyethylene (HDPE) used for moving bed biofilm reactor (MBBR). During treatment, the plastic media are expose to a dust of silica particles, such as silica microspheres (FIG. 1). The process is at a temperature comprised between 130-170° C. for a duration between 15 min and 2 h. After cooling down, silica microspheres are trapped on the plastic surface which create silica particles covered plastic media (FIG. 3B). The visual comparison of the media expose to silica deposition shows a good visual difference between a plastic media a treated and non-treated media (FIG. 3C). Scanning electronic microscopy confirm the deposition (FIGS. 2A and 2B).

### Example 2

### Silica Particles (Powder) Deposition During Plastic Material Production

An example of silica particles deposition during plastic material production. Plastic media used for MBBR are fabricated by extrusion. The extrusion process, which is a mechanical and thermal process, is modified in such a manner that silica particles is deposited on the surface of the plastic media during the process. Silica particles are sprayed when the plastic material exits the extruder during the cooling process.

### Example 3

### Silica Particles (Slurry) Deposition During Plastic Extrusion Process

An example of silica particles deposition during plastic material production. Plastic media used for MBBR are fabricated by extrusion. The extrusion process, which is a mechanical and thermal process, is modified in such a manner that silica particles is deposited on the surface of the plastic media during the process. The extruded plastic is soaked in a hot slurry containing the silica slurry and deposition occur during that step.

### Example 4

### Silica Particles Coated Plastic Added Value in IFAS

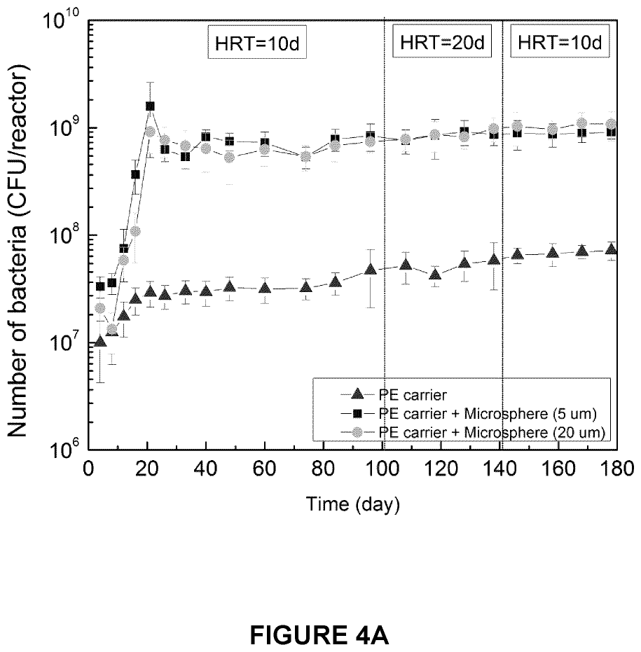

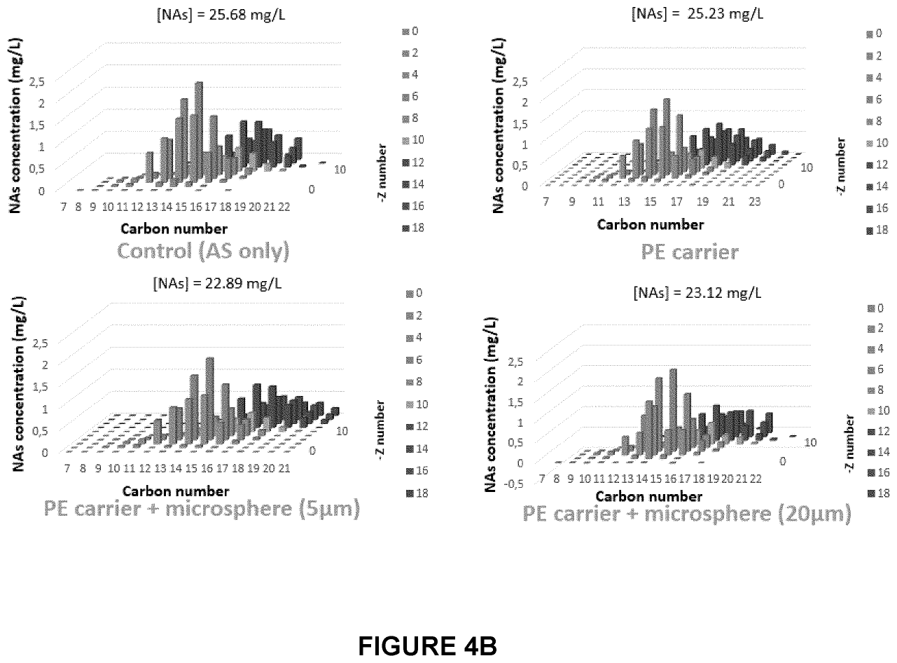

An example of the silica particles coated plastic added value. Plastic media covered with silica particles are used at laboratory scale to validate the effect of the added surfaces property. It is believed that adding particles to plastic would add more specific surface which would increase microbial adhesion. It is also hypothesized that the functionalized silica would increase interaction between the surface and the bacteria. Thus, test in Erlenmeyer are performed to evaluate if the density of the microbial population can be increased. The tests are performed in relation to oil sand tailing pond biological treatment. The experimental condition are as follow: reactor volume: 500 ml; plastic media per reactors: 50; hydraulic retention time: 10 days; dissolve oxygen: 6-7 mg/L; chemical oxygen demand to nitrogen ration: 11.7; days of operation: 180 days; chemical oxygen demand: 350 mg/L. Bacteria population, shown as count bacteria enumeration (CFU), for the different evaluated treatments are shown in FIG. 4A. The three-evaluated treatment are: plastic media with no treatment (PE carrier), plastic media covered with 5 μm silica microspheres (PE carrier+microspheres 5 μm), plastic media covered with 20 μm microspheres (PE carrier+microspheres 20 μm). Results demonstrate highly significant increases of bacterial population on plastic media covered with microspheres (FIG. 4A). The Erlenmeyer were operated to simulate an Integrated Fixed-Film Activated Sludge (IFAS) process and samples were taken at the end of the experiment to monitor naphthenic acid (NA) treatment. Results demonstrate that adding regular plastic media to an activated sludge reactor does not lead to lower concentration of NA compared to activated sludge alone (25.7 compared to 25.2 mg/ml, FIG. 4B). On the contrary, adding plastic media covered with silica particles lead to statistically significant lower concentration of NA than both activated sludge alone and activated sludge combined with regular plastic media (25.2 compared to 23.1 and 22.9 mg/ml FIG. 4B).

### Example 5

### Silica Particles Coated Plastic Added Value in MBBR

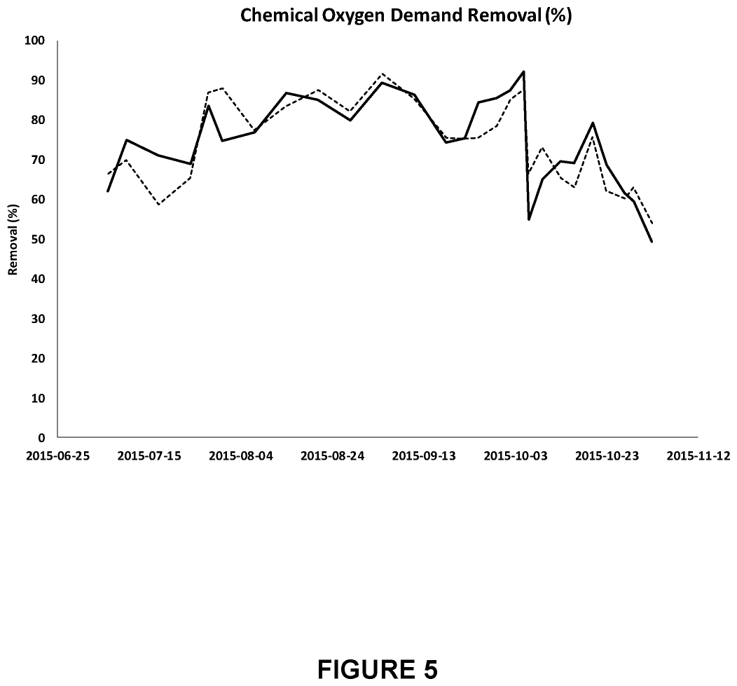

An example of the silica particles coated plastic added value. Plastic media covered with silica particles are tested in bench test MBBR to validate that added particles does not change operation conditions. Adding matter to plastic media could change the media density which may alter proper operation of MBBR. The influent was as follow: soluble chemical oxygen demand: 20 to 150 ppm; total soluble phosphorus: 0.7 to 2.5 ppm, total soluble (kjeldahl) nitrogen: 11.2 to 24 ppm; pH 7.20 to 8.10. The hydraulic retention time was varied from 8 to 1 h. Results demonstrated that the reactor can be operated with the same parameter. Non-optimal operation shows that similar treatment is achieved (FIG. 5).

### Example 6

### Silica Particles Deposition During Plastic Film Production by Extrusion

An example of silica particles deposition during plastic material production. An extrusion process produces thin sheets of plastic. The sheet of plastic coming out of the extruder are exposed to a flow of silica particles. The silica particles are deposited on the plastic before the plastic hardens.

### Example 7

### Silica Particles Coated Plastic Added Value in Aerated Pond

An example of the silica particles coated plastic added value. Plastic sheet such as produces in example 6 are plunged under water, such as in an aerated pond related to wastewater treatment or in a lake, river or pond under biological treatment. The plastic sheets serve as support media for the growth of bacteria. The technology would generally be used along with other equipment such as aeration devices.

### Example 8

### Silica Particles Deposition on Plastic Dropplet in a Hot Slurry

An example of silica particles deposition during plastic material production. Melted plastic material is introduced as plastic droplet into a stirred tank containing a hot slurry. The slurry temperature is initially above the plastic melting point. Plastic is introduced into the tank until its volume fraction reaches about 10%. Once the plastic droplets addition operation is completed, the temperature is lowered slowly from above the melting point to under the melting point which allows the droplets to solidify with silica particle covering its surfaces. The droplets then become plastic beads covered with silica particle. Once the slurry reaches a certain temperature corresponding to the bead being solid enough for further manipulation, the agitation is stopped, and the slurry is separated from the bead using a grid. The slurry is recycled for the next batch and the bead are taken out for subsequent washing steps.

### Example 9

### Silica Particles Coated Plastic Added Value in Column Reactor

An example of the silica particles coated plastic added value. A compound “C” is to be removed from a stream of liquid. One way to do it is by adsorption of the compound using an adequate adsorbent. The industrial method to use adsorption is through the use of column packed with the adsorbent. A column packed with plastic beads covered with silica particle such as described in Example 8 is used to capture the compound “C”. The bead diameter is sufficiently large to allow the liquid to flow from top to bottom by using gravity. The compound “C” is adsorbed due to the silica high surface area.

### Example 10

### Silica Particles Coated Plastic Added Value in Enzymatic Column Reactor

An example of the silica particles coated plastic added value. An enzymatic process required the substrate S to be converted into the product P by the enzyme E. The reaction is a continuous process done in a packed-bed column reactor. The column reactor is packed with plastic bead covered silica such as described in Example 8. The silica covering the beads are mesoporous functionalized silica microspheres that are used for enzyme immobilization. Before being placed into the column, the bead had been put in contact with enzyme and the enzyme has been immobilized on the silica surface. When in operation, the packed column continuously receives a stream of liquid containing the substrate. As the stream progress through the column, the substrate is converted by the enzyme into the product. The outlet of the column supplies a continuous flow of product. The liquid progress though the column by gravity, from the top inlet to the bottom outlet.

### Example 11

### Silica Particles Coated Plastic Added Value in MBBR—Second Example

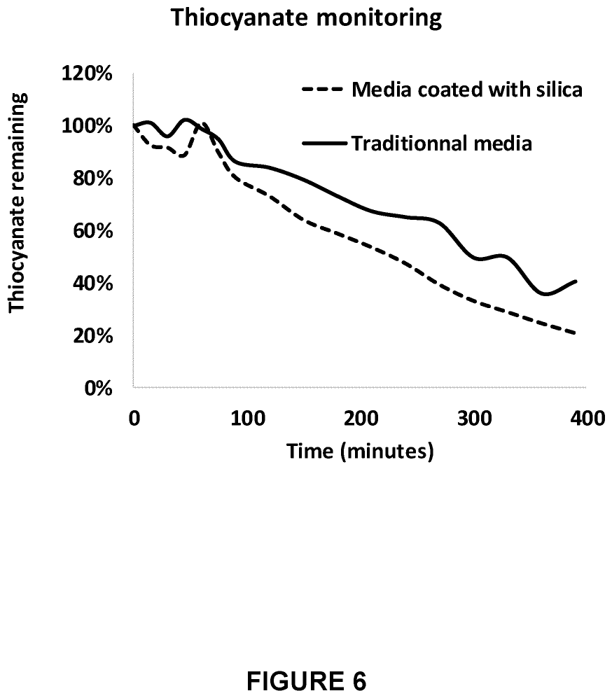

An example of the silica particles coated plastic added value. In Example 5, it was demonstrated that moving bed biofilm reactor (MBBR) with plastic media coated with silica microsphere can be operated similarly to MBBR usual traditional media. In this example, we want to demonstrate the performance gains that can be obtain for such reactor. In order to simulate the plastic media replacement of a large 140 m³ MBBR reactor, a small-scale experiment was done to evaluate the increases of performances. Four liters of traditional plastic media were put into a net; the net was then placed into an already operating 140 m³ MBBR reactor for a month in order to give time for the plastic media to be colonized by the bacterial flora of the reactor. The same was done for plastic media coated with microspheres. The two nets were then taken out of the reactor at the same time. Then, the media of each net were place into a bucket of influent wastewater. Thiocyanates measurement were done by interval of 15 minutes for over 6 hours to monitor the thiocyanates consumption by the bacteria immobilized on the plastic media. The initial thiocyanate concentration was around 250 ppm in the influent. After 6 hours, 41% of the thiocyanates were remaining in the bucket containing the traditional media while only 20% of the thiocyanate remaining in the bucket containing the media coated with silica. The thiocyanate monitoring can be found in FIG. 6 of the drawing.

### Example 12

### Silica Particles Coated Plastic Added Value in Tailing Pond

An example of the silica particles coated plastic added value. In order to increase the biological remediation of oil sand process water (OSPW) tailing pond, plastic media coated with silica according to the present invention are placed into several floating islands whose purpose is to favor bacterial development which would treat the OSPW. The floating island consist of a mean to retain the plastic media and ensure the media are placed just below the water surface.

### Example 13

### Silica Particles Coated Plastic Added Value in Tailing Pond—Second Example

An example of the silica particles coated plastic added value. In order to increase the biological remediation of oil sand process water (OSPW) tailing pond, an artificial river has been created to treat the effluent of the tailing pond. The river has been designed similarly to rivers that are layed out to promote oxygenation for fish such as trouts; rocks are placed in rapids in order to favor oxygenation and a pit has been placed. In this artificial river, plastic media coated with silica are found in the pit and are retained by grid and netting. Alternatively, floating island using plastic media coated with silica could be used. The artificial river has the same function as the moving bed biofilm reactor (MBBR): oxygenation and water flow. Thus, the artificial river is a passive treatment system that required no pump and no air blower. Oxygenation zone and treatment zone are alternated in the river and water pollutants thus decrease from the upstream to the downstream.

### Example 14

### Silica Particles Coated Plastic Added Value for Fast Reactor Start-Up

An example of the silica particles coated plastic added value. The ANAMMOX process used in the field of biological wastewater treatment have long start-up time ranging from 8 months to 1.5 years. Various strategy has been employed to reduce the start-up time such as seeding the reactor with plastic media colonized by ANAMMOX flora or seeding the reactor with activated sludge. The present invention allows a much faster media colonization, and as such could be very well being part of a global strategy for faster ANAMMOX reactor start-up. Fresh plastic media coated with siliceous particles could be quickly colonized by an ANAMMOX flora already existing in the environment or being seeded into the reactor.

### Example 15

### Silica Particles Coated Plastic Added Value to Introduce Specific Microorganisms Flora into a New Environment

An example of the silica particles coated plastic added value. In some applications, such a biological wastewater treatment, it is sometimes desirable to introduce a specific microbial population to achieve a specific metabolic conversion. For instance, if there is a need to treat a specific pollutant by biological treatment and the bacterial flora is not able to do so; then it is required to develop a new bacteria consortium which is able to degrade the specific pollutant. However, it is frequent that the new consortium would be unable to colonize its new environment. The difficulty arises from the competition between the newly arrived microbial population and the already established one(s); in many cases, the new populations won't be able to compete and will be washed out of the new environment. One way to overcome this problem is to bring a fixed microbial culture into that new environment; this option is carried out through the use of plastic media support. The difficulty of producing fixed culture on plastic media is that plastic media takes too long to be colonized. An easy way to bring a consortium into an environment would be to introduce a plastic media coated with siliceous particles into the bioreactors already producing the consortium; the modified media would be colonized during fermentation which would not be possible with a non-coated media due to long colonization time. The colonized media could then be dried, stored and incubated when needed.

### Example 16

### Silica Particles Coated Plastic Added Value for Fast Reactor Start-Up

An example of the silica particles coated plastic added value. In wastewater treatment, process start-up time is a value to be minimized. For specific application known to the art, there is great benefits into reducing the media colonization time. One way to achieve such a challenge would be to introduce the plastic media that are already inoculated with a microorganism's flora such as described in Example 15.

### Example 17

### Silica Particles Coated Plastic Added Value in Enzymatic Column Reactor

An example of the silica particles coated plastic added value. Commercial Laccase from Trametes versicolor (Sigma Aldrich) was used in these experimental sets. Constant concentration of glutaraldehyde (GLU) was used for the immobilization process (1 ml 25% wt aqueous Glutaraldehyde for each 10 ml samples). After 1 ml GLU added in 10 ml buffer solutions, the system was conditioned with all supports (plastic packing, plastic packing-silica, silica powder) for 12 hours with supports.

Enzyme concentration was selected by measuring approximately amount of silica on packing to always have the same enzyme to silica packing ratio. Randomly selected 45 packings and silicate packing were weighed and differences amount of these average packing weight were assumed as silica amount which is integrated with packing. 0.5 mg enzyme was used per mg silica.

The Immobilized Laccase Activity yield show after the 3 days that the plastic packing without the silica treatment have a yield below 50% while the plastic packing with the silica is close to 100%.

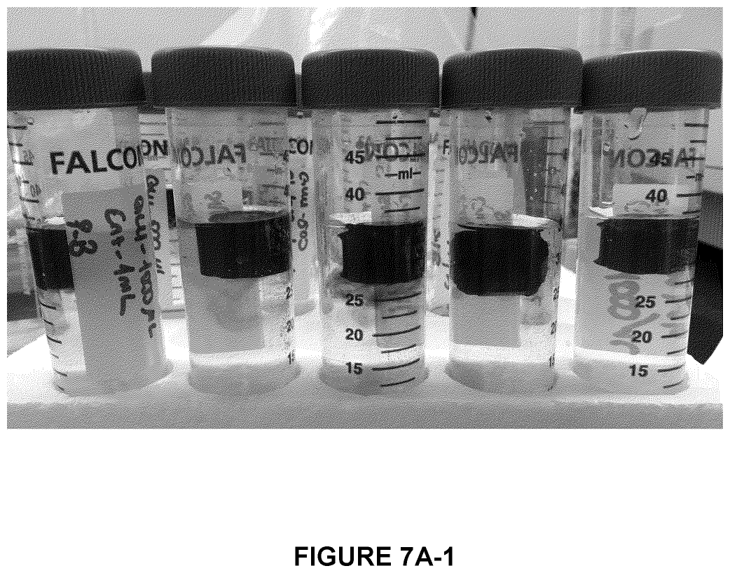

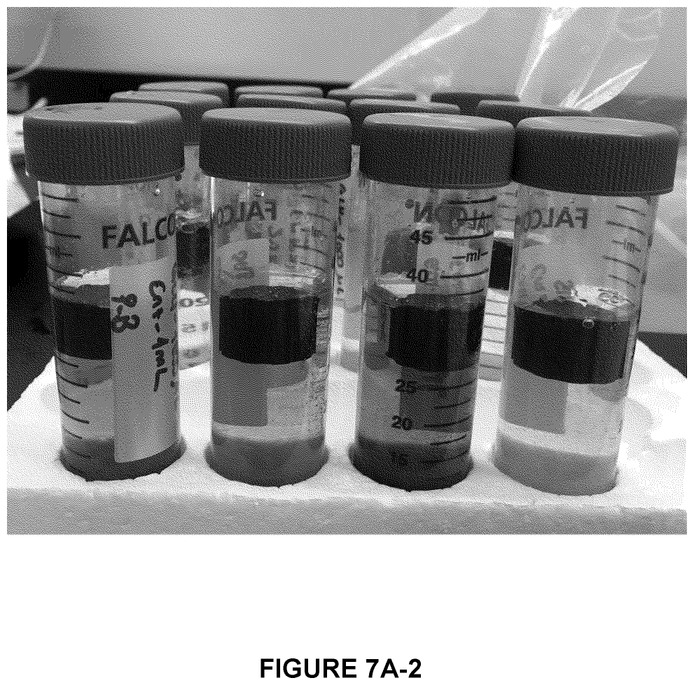

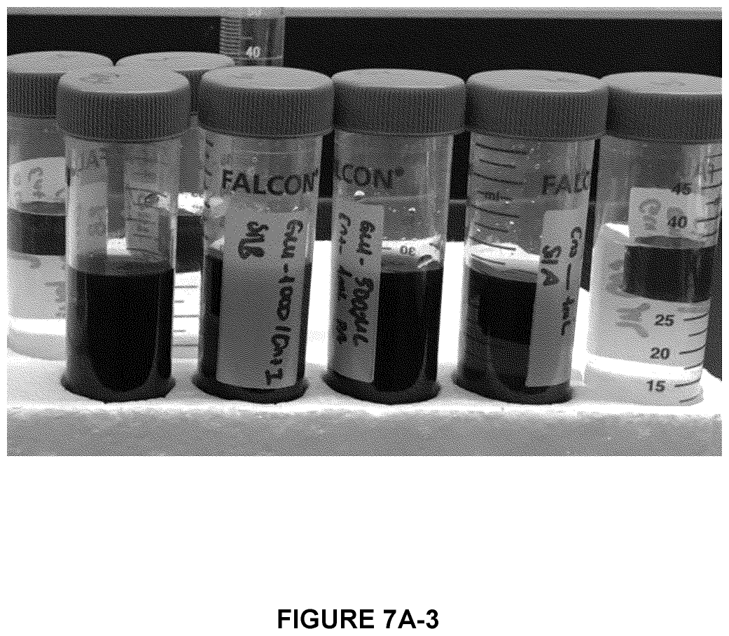

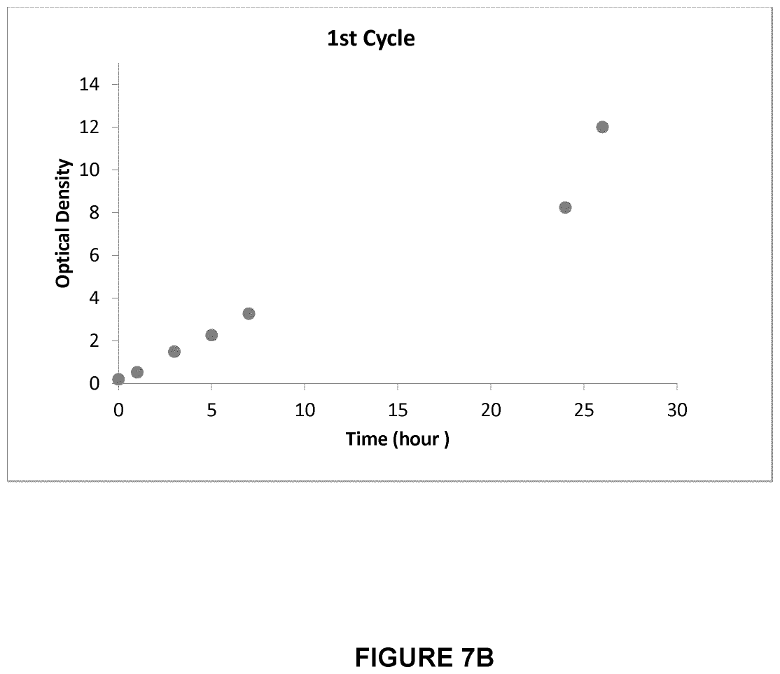

Evaluation of the enzymatic activity of individual plastic media coated with microspheres were evaluated in a lab experiment. The plastic media were place in a solution containing ABTS (2,2′-azino-bis(3-ethylbenzothiazoline-6-sulphonic acid) which is converted to a colored product by the laccase. At time zero the solution is clear, and it is observed that a colored product beginning to form on the surface of the plastic media (FIG. 7A-1); after 30 minute the liquid has turned to light green (FIG. 7A-2); after 4 hours, the liquid has turned dark green due to continuous conversion of ABTS to the colored product (FIG. 7A-3). For a single plastic media, conversion of ABTS is monitored over 26 hours; it is observed that the enzymatic activity is stable over all the 26 hours of the test since the optical density rises at a constant level (FIG. 7B); the same plastic media was tested for 10 cycles and over the 10 cycles constant activity was maintained.

### Example 18

### Silica Particles Coated Plastic Added Value in Adsorption of Emerging Contaminants

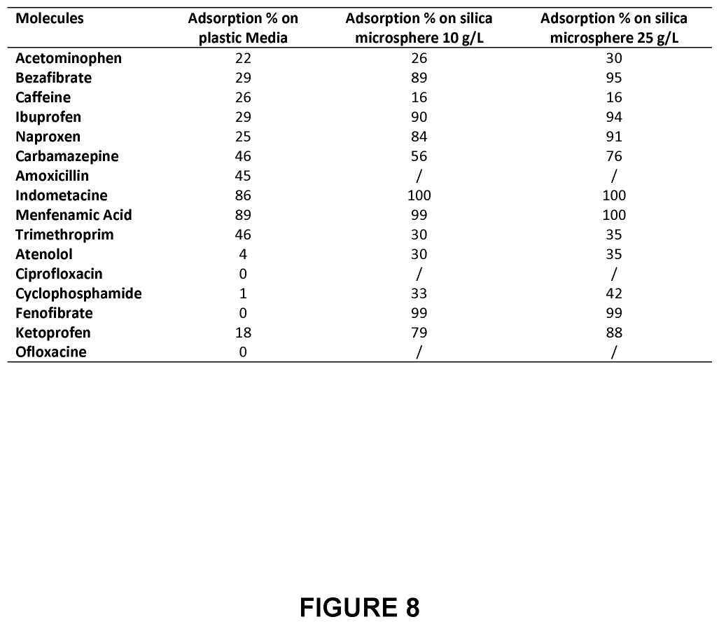

An example of the silica particles coated plastic added value. The plastic media coated with silica has been tested in the application of adsorption of Emerging contaminants. The plastic media was put in contact with 16 emerging contaminants. The applied concentration for the adsorption tests are 100 μg/L per contaminant in 30 mL at pH 6.5 using 1 plastic media. At the same time the silica microspheres have been tested without plastic media using two different concentrations 10 g/L and 25 g/L. The results of this experiment are shown at the FIG. 8. The list of contaminants is as follow: Acetominophen, Bezafibrate, Caffeine, Ibuprofen, Naproxen, Carbamazepine, Amoxicillin, Indometacine, Menfenamic Acid, Trimethroprim, Atenolol, Ciprofloxacin, Cyclophosphamide, Fenofibrate, Ketoprofen, Ofloxacine. The results show that the plastic media coated with silica have a suitable adsorption capacity with the emerging contaminants.

### Example 19

### Silica Particles Coated Plastic Added Value in Biological Oxygen Demand

An example of the silica particles coated plastic added value. The plastic media coated with silica has been tested for the growth of a bio-film and the consumption of a biofilm. The plastic media has been exposed to a synthetic waste water containing 3 g/L of Dextrose and 1 g/L of powdered milk mixed with a bacterial consortium. The water was changed every two days and the new water was containing the same concentration of nutrients. The total duration for the bio-film growth was 4 weeks. In the last day a kinetic study of the sugar consumption is performed. The initial concentration of the reducing sugar was 3 g/L. The results are presented in the following table. The dosing of the reducing sugar has been done using Benedict's method. The results show that the presence of a thicker biofilm induces the consumption of higher amount of sugar by the bacteria.

| | Sugar consumption % | | | |
|---|---|---|---|---|
| | Average | RSD | Average | RSD |
| Sample | 4 h | | 7 h | |
| Plastic media without silica | 92% | 3% | 82% | 3% |
| Plastic media with silica A | 65% | 3% | 39% | 6% |
| Plastic media with silica B | 63% | 2% | 33% | 4% |
| Plastic media with silica C | 65% | 12% | 44% | 11% |
| Plastic media with silica D | 64% | 1% | 36% | 9% |

The difference between silica A, silica B, silica C and silica D is the shape of the particles. All the media with silica perform better than a media without silica.

While preferred embodiments have been described above and illustrated in the accompanying drawings, it will be evident to those skilled in the art that modifications may be made without departing from this disclosure. Such modifications are considered as possible variants comprised in the scope of the disclosure.

1. Surface modified polymeric material comprising a plurality of silica particles deposited and partially embedded on a surface thereof, wherein said silica particles are bioavailable for interaction with a microorganism or a biological molecule or complex, available for chemical interaction, available for chemical reaction, or a combination thereof.

2. The surface modified polymeric material of claim 1, wherein said plurality of silica particles is a plurality of one type of silica particle, a plurality of at least one type of silica particle, or a plurality of more than one type of silica particle.

3. The surface modified polymeric material of claim 2, wherein said polymeric material is a plastic material.

4. The surface modified polymeric material of any one of claims 1-3, wherein said plurality of silica particles deposited and partially embedded on a surface thereof is deposited on said surface is at or over a melting point of said polymeric material.

5. The surface modified polymeric material of any one of claims 1-4, wherein said silica particles are about 10% to about 90% partially embedded in the polymeric material.

6. The surface modified polymeric material of any one of claims 1-5, wherein said silica particles cover from about 0.01% to 100% of said surface.

7. The surface modified polymeric material of any one of claims 1-6, wherein said silica particle is a nanoparticle, a microparticle, a nanosphere, a microsphere, or combinations thereof.

8. The surface modified polymeric material of any one of claims 1-7, wherein said silica particles have a diameter of from about 10 nm to about 15 mm or a combination thereof.

9. The surface modified polymeric material of any one of claims 1-8, wherein said silica particles are crystalline silica or amorphous silica.

10. The surface modified polymeric material of any one of claims 1-9, wherein said silica particles are spherical particles or of a random geometry.

11. The surface modified polymeric material of any one of claims 2-10, wherein said silica particles are hollow particles or full particles.

12. The surface modified polymeric material of any one of claims 2-11, wherein said silica particles are porous or non-porous.

13. The surface modified polymeric material of any one of claims 2-12, wherein said silica particles comprise a chemical functional group.

14. The surface modified polymeric material of claim 13, wherein said chemical functional group is available for said chemical reaction and/or chemical interaction.

15. The surface modified polymeric material of any one of claims 2-14, wherein said silica particles are covered with an allotrope of carbon.

16. The surface modified polymeric material of any one of claims 2-15, wherein said silica particles are covered with metallic particles or a coating.

17. The surface modified polymeric material of claim 16, where said coating is a metals salt coating, a metal oxide coating, an organometallic coating, an organic coating.

18. The surface modified polymeric material of claim 17, where said organic coating is a polymer, a biopolymer or a combination thereof.

19. The surface modified polymeric material of any one of claims 2-18, wherein said silica particles are covered or coated with a microorganism.

20. The surface modified polymeric material of claim 19, wherein said microorganism is a bacteria, a fungi, a yeast, a mold, a spore, a filament, a gram negative bacteria, a gram positive bacteria, a dried microorganism, a microfilm supporting microorganism in a growth ready state, a vegetative state microorganism.

21. The surface modified polymeric material of claim 19, wherein said vegetative state microorganism is synchronized and arrested in a specific phase of life cycle, arrested in a specific phase of life cycle, not synchronized and arrested in a specific phase of life cycle, not synchronized in a specific growth phase, ready to be activated in the presence of a suitable carbon source, or a combination thereof.

22. The surface modified polymeric material of any one of claims 2-21, wherein said silica particles have encapsulated, adsorbed and/or absorbed a chemical, a biologically active molecule, or a combination thereof.

23. The surface modified polymeric material of claim 22, wherein said biologically active molecule comprises an enzyme, a hormone, an antibody or a functional fragment thereof, a bio suppressant, or combinations thereof.

24. The surface modified polymeric material of claim 23, wherein said chemical comprises an antibiotic, an anti-viral, an anti-toxin, a pesticide, or combinations thereof.

25. The surface modified polymeric material of any one of claims 2-21, wherein said silica particles is a silica shell having a thickness of from about 50 nm to about 500 μm, and a plurality of pores, said shell forming a capsule having a diameter from about 0.2 μm to about 1500 μm, and having a density of about 0.01 g/cm³ to about 1.0 g/cm³,
wherein said shell comprises from about 0% to about 70% Q3 configuration, and from about 30% to about 100% Q4 configuration, or
wherein said shell comprises from about 0% to about 60% T2 configuration and from about 40% to about 100% T3 configuration, or
wherein said shell comprises a combination of T and Q configurations thereof, and
wherein an exterior surface of said microcapsule is covered by a functional group.

26. The surface modified polymeric material of claim 25, wherein said shell comprises about 40% Q3 configuration and about 60% Q4 configuration, or about 100% Q4 configuration.

27. The surface modified polymeric material of any one of claims 25-26, wherein said pores have pore diameters from about 0.5 nm to about 100 nm.

28. The surface modified polymeric material of any one of claims 25-27, further comprising a surface layer.

29. The surface modified polymeric material of any one of claims 25-28, wherein said surface layer comprises a thickness from about 1 nm to about 10 nm.

30. The surface modified polymeric material of any one of claims 25-26, wherein said surface layer is functionalized with an organosilane.

31. The surface modified polymeric material of claim 30, wherein said organosilane is chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

32. The surface modified polymeric material of claim 30, wherein said surface layer is functionalized with a hydroxyl group, an amino group, a benzylamino group, a chloropropyl group, a disulfide group, an epoxy group, a mercapto group, a methacrylate group, a vinyl group, and combinations thereof.

33. A product prepared with the surface modified polymeric material of any one of claims 1 to 32.

34. The product of claim 33, wherein said product is a sheet of polymeric material, a polymeric material droplet or bead, a polymeric material media for use in wastewater treatment.

35. The product of any one of claims 33-34, wherein one or more surface of said product comprises said plurality of silica particles deposited and partially embedded thereof.

36. A process for the preparation of a surface modified polymeric material comprising a plurality of silica particles deposited and partially embedded on a surface thereof, the process comprising the step of:
contacting a surface of polymeric material at a temperature at or over a melting temperature of said polymeric material with a plurality of silica particles, wherein said silica particles are deposited and partially embedded thereon, and are bioavailable for interaction with a microorganism or a biological molecule or complex, available for chemical interaction, available for chemical reaction, or a combination thereof.

37. The process of claim 36, wherein said plurality of silica particles is a plurality of one type of silica particle, a plurality of at least one type of silica particle, or a plurality of more than one type of silica particle.

38. The process of any one of claims 36-37, wherein said polymeric material is a plastic material.

39. The process of any one of claims 36-38, wherein said silica particles are deposited and partially embedded in said polymeric material by a mechanical treatment, thermal treatment, chemical treatment, or a combination thereof.

40. The process of any one of claims 36-39, wherein said silica particles are deposited and partially embedded during the polymeric material production process by an extrusion process, an injection process, a thermoforming, a compression molding, a rotational molding, a blow molding, a pultrusion, or combinations thereof.

41. The process of any one of claims 36-40, wherein said polymeric material is provided as droplets.

42. The process of any one of claims 36-41, wherein said silica particles are deposited and partially embedded after the polymeric material production process.

43. The process of any one of claims 36-42, wherein said silica particles are deposited and partially embedded in the plastic using heat supplied by convection, conduction or radiation.

44. The process of any one of claims 36-43, wherein said polymeric material is heated to a temperature at or over a melting temperature of said polymeric material provided by a hot air or gas, a flame, a hot slurry, a hot liquid, a sonication, a mechanical wave, a plasma, an electricity, a lamp, a heating element, a conductive plate, or combinations thereof.

45. The process of any one of claims 36-44, wherein said silica particles are deposited or partially embedded as a suspended powder, as a slurry, or a combination thereof.

46. The process of any one of claims 36-45, wherein said silica particles are about 10 to about 90% partially embedded in the polymeric material.

47. The process of any one of claims 36-46, wherein said silica particles cover from about 0.01% to 100% of said surface.

48. The process of any one of any one of claims 36-47, wherein said silica particle is a nanoparticle, a microparticle, a nanosphere, a microsphere, or combinations thereof.

49. The process of any one of claims 36-48, wherein said silica particles have a diameter of from about 10 nm to about 10 mm or a combination thereof.

50. The process of any one of claims 36-49, wherein said silica particles are crystalline silica or amorphous silica.

51. The process of any one of claims 36-50, wherein said silica particles are spherical particles or of a random geometry.

52. The process of any one of claims 36-51, wherein said silica particles are hollow particles or full particles.

53. The process of any one of claims 36-52, wherein said silica particles are porous or non-porous.

54. The process of any one of claims 36-53, wherein said silica particles comprise a chemical functional group.

55. The process of any one of claims 36-54, wherein said chemical functional group is available for said chemical reaction.

56. The process of any one of claims 36-88, wherein said silica particles are covered with an allotrope of carbon.

57. The process of any one of claims 36-56, wherein said silica particles are covered with metallic particles or a coating.

58. The process of claim 57, where said coating is a metals salt coating, a metal oxide coating, an organometallic coating, an organic coating.

59. The process of claim 58, where said organic coating is a polymer, a biopolymer or a combination thereof.

60. The process of any one of claims 36-59, wherein said silica particles are covered or coated with a microorganism.

61. The process of any one of claims 36-55, wherein said microorganism is a bacteria, a fungi, a yeast, a mold, a spore, a filament, a gram negative bacteria, a gram positive bacteria, a dried microorganism, a microfilm supporting microorganism in a growth ready state, a vegetative state microorganism.

62. The process of claim 61, wherein said vegetative state microorganism is synchronized and arrested in a specific phase of life cycle, arrested in a specific phase of life cycle, not synchronized and arrested in a specific phase of life cycle, not synchronized in a specific growth phase, ready to be activated in the presence of a suitable carbon source, or a combination thereof.

63. The process of any one of claims 36-62, wherein said silica particles have encapsulated, adsorbed or absorbed a chemical, a biologically active molecule, or a combination thereof.

64. The process of claim 63, wherein said biologically active molecule comprises an enzyme, a hormone, an antibody or a functional fragment thereof, a bio suppressant, or combinations thereof.

65. The process of claim 63, wherein said chemical comprises an antibiotic, an anti-viral, an anti-toxin, a pesticide, or combinations thereof.

66. The process of any one of claims 36-65, wherein said silica particles is a silica shell having a thickness of from about 50 nm to about 500 μm, and a plurality of pores, said shell forming a capsule having a diameter from about 0.2 μm to about 1500 μm, and having a density of about 0.01 g/cm³ to about 1.0 g/cm³,
wherein said shell comprises from about 0% to about 70% Q3 configuration, and from about 30% to about 100% Q4 configuration, or
wherein said shell comprises from about 0% to about 60% T2 configuration and from about 40% to about 100% T3 configuration, or
wherein said shell comprises a combination of T and Q configurations thereof, and
wherein an exterior surface of said microcapsule is covered by a functional group.

67. The process of claim 66, wherein said shell comprises about 40% Q3 configuration and about 60% Q4 configuration, or about 100% Q4 configuration.

68. The process of any one of claims 66-67, wherein said pores have pore diameters from about 0.5 nm to about 100 nm.

69. The process of any one of claims 66-68, further comprising a surface layer.

70. The process of any one of claims 66-69, wherein said surface layer comprises a thickness from about 1 nm to about 10 nm.

71. The process of any one of claims 66-70, wherein said surface layer is functionalized with an organosilane.

72. The process of claim 71, wherein said organosilane is chosen from a functional trimethoxysilane, a functional triethoxysilane, a functional tripropoxysilane, 3-aminopropyltriethoxysilane, vinyltriacetoxy si lane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

73. The process of any one of claims 66-72, wherein said surface layer is functionalized with a hydroxyl group, an amino group, a benzylamino group, a chloropropyl group, a disulfide group, an epoxy group, a mercapto group, a methacrylate group, a vinyl group, and combinations thereof.

74. A method for the treatment of wastewater or a contaminated soil, comprising contacting wastewater or contaminated soil with a surface modified polymeric material of any one of claims 1-32, a product according to any one of claims 33-35, or combinations thereof, for a time sufficient and under conditions sufficient for decontaminating said wastewater or contaminated soil.

75. The method of claim 74, wherein said treatment of wastewater is in a moving bed biofilm reactor (MBBR), an Integrated Fixed-Film Activated Sludge (IFAS) reactor, an aerated pond, a non-aerated pond, a membrane bioreactor (MBR), a sequential batch reactor (SBR), a water polishing processes, with an activated sludge, or combinations thereof.

76. The process of any one of claims 74-75, wherein said surface modified polymeric material or said product is a media for use in wastewater treatment.

77. A biological process comprising contacting a culture media with a surface modified polymeric material of any one of claims 1-32, a product according to any one of claims 33-35, or combinations thereof for a time sufficient and under conditions sufficient for any one of a fermentation, a pre-culture, a media preparation, a harvesting of a product, concentration of a product, purification of a product.

78. A process comprising contacting a solution with a surface modified polymeric material of any one of claims 1-32, a product according to any one of claims 33-35, or combinations thereof under conditions sufficient to perform a reaction or an interaction with said surface modified polymeric material and/or said product.

79. The process of claim 48, wherein said process is performed in a column.

80. The process of any one of claims 78-79, wherein said process is a chromatography, an adsorption, a catalysis, or combinations thereof.

81. The process of any one of claims 78-79, wherein said process is an enzymatic process.
